(19) United States
(12) Patent Application Publication
(10) Pub. No.: US 2006/0287535 A1
(43) Pub. Date: Dec. 21, 2006

(54) Process for the manufacture of 1,2-benzisoxazole-3-methanesulphonamide

(75) Inventors: Siddiqui Mohammed Jaweed Mukarram, Aurangabad (IN); Aravind Yehanathsa Merwade, Aurangabad (IN); Jagdish Dattopant Shukla, Aurangabad (IN); Anis Mushtaqeali Saiyad, Aurangabad (IN)

Correspondence Address: IPLM GROUP, P.A., POST OFFICE BOX 18455, MINNEAPOLIS, MN, 55418, US

(73) Assignee: Wockhardt Limited

(21) Appl. No.: 11/432,673

(22) Filed: May 11, 2006

(30) Foreign Application Priority Data
Nov. 11, 2003 (WO) PCT/IB03/05052
May 19, 2005 (WO) 2005/044808

(51) Int. Cl. C07D 261/20

(52) U.S. Cl. 548/241

(57) ABSTRACT

The present invention provides an improved process for the preparation of 1,2-Benzisoxazole-3-methanesulphonamide (Zonisamide), an anti-epileptic agent having anti-convulsant and anti-neurotoxic effects. In another aspect, the invention provides a key intermediate, 1,2-benzisoxazole-3-methane sodium sulfonate (BOS-Na:NaCl), isolated as a crystalline sodium chloride associated compound. The BOS-Na:NaCl isolated is directly converted to Zonisamide using a controlled molar ratio of chlorosulfonic acid to avoid conversion to the disulfonated side products.

### CROSS-REFERENCE TO RELATED APPLICATION

This application claims priority from international patent application Serial No. PCT/IB2003/005052, filed Oct. 28, 2003, and published in English on May 19, 2005 as International Publication No. WO 2005/044808 A1, which are incorporated herein by reference.

### FIELD OF THE INVENTION

The present invention relates to an improved process for the manufacture of 1,2-Benzisoxazole-3-methanesulphonamide (Zonisamide) which is known as an anti-epileptic agent which possesses anti-convulsant and anti-neurotoxic effects. Another aspect of the invention is the preparation of crystalline 1,2-benzisoxazolemethane sulphonic acid sodium salt (BOS-Na) associated with sodium chloride and its subsequent conversion of BOS-Na to Zonisamide. The physical properties such as infra-red absorption values and X-ray Powder Diffraction data of the new intermediate, i.e., sodium chloride associated 1,2-benzisoxazole methanesulphonic acid sodium salt are reported to confirm its identity. The Zonisamide obtained using the synthesized intermediate has good purity.

### BACKGROUND OF THE INVENTION

1,2-Benzisoxazole-3-methanesulphonamide (chemical name) is commercially known as Zonisamide. It has the chemical structure shown in Formula I:

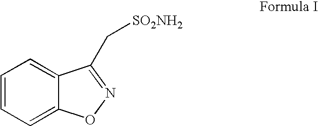

Zonisamide is an anti-epileptic agent which possesses anti-convulsant and anti-neurotoxic effects. Several routes for the manufacture of Zonisamide are reported in the literature. For example, procedures for the preparation of Zonisamide are disclosed in U.S. Pat. No. 4,172,896; J. Heterocyclic Chem. 6, 279 (1969); Ibid, 8, 397 (1971); U.S. Pat. No. 4,172,896; and Japanese Patent 53-77057, assigned to Dainippon Pharmaceutical Co. These synthetic routes involve the preparation of 1,2-benzisoxazolemethanesulphonyl chloride, an intermediate used in the Zonisamide synthesis.

The first route for the synthesis of Zonisamide beginning with the bromination of benzisoxazole acetic acid (BOA), followed by substitution of the bromine using sodium sulfite (to provide the intermediate sodium salt of benzisoxazole methane sulphonic acid (BOS-Na)) is shown in Scheme I, below:

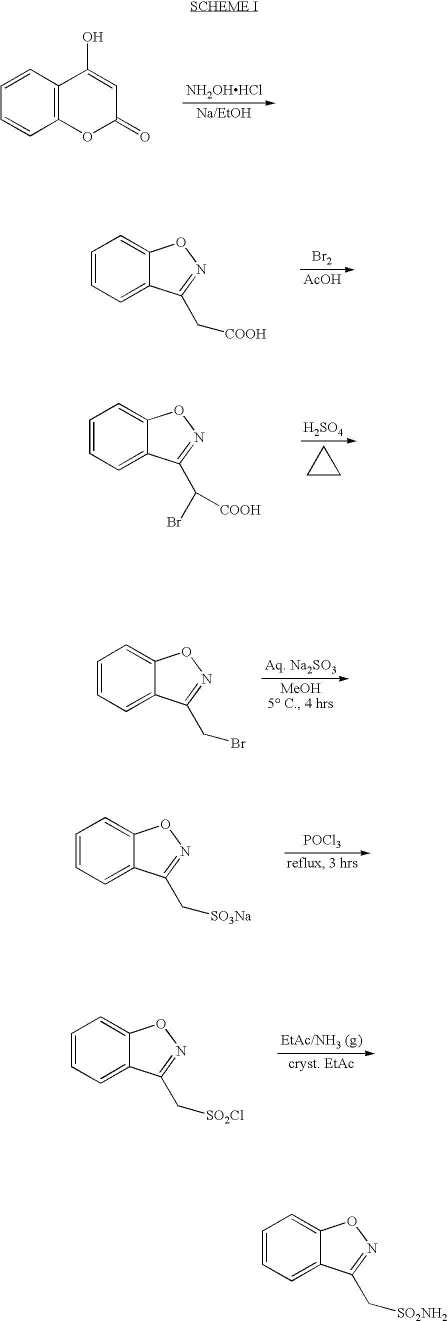

The manufacturing process for Zonisamide disclosed in Scheme I is difficult to reproduce because it requires the use of bromine and the intermediate product is very sensitive.

An alternative manufacturing process for Zonisamide, also starting with 4-hydroxy-coumarin, and preparing the same intermediates, 1,2-Benzisoxazole acetic acid and BOS-Na as used in Scheme I, is shown in Scheme II, below:

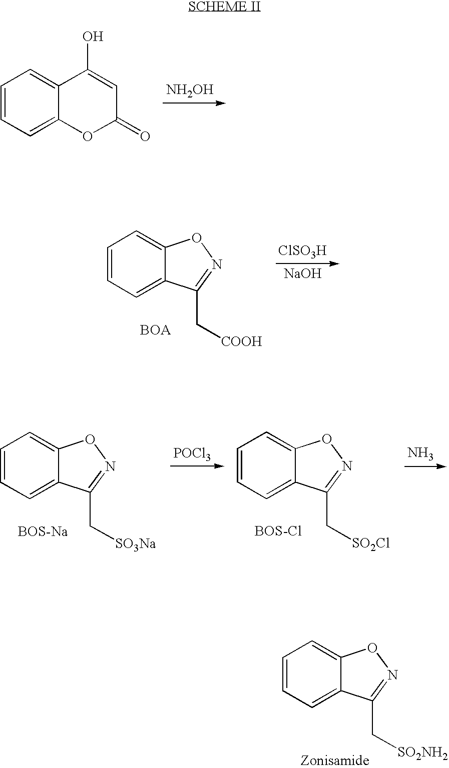

BOA is converted to the BOS-Na in reaction with ClSO₃H/dioxane in ethylene chloride, at room temperature for about 3 hours, followed by heating at 50° C. for 6 hours. After the reaction is complete, water and sodium hydroxide are added and the product is isolated as sodium salt (BOS-Na) by evaporation of the aqueous layer. Using BOS-Na, Zonisamide has been synthesized as discussed in the process disclosed above.

U.S. Patent Application No. 2002/0183525 A1 (“the '325 application”) discloses a process for preparing 1,2-Benzisoxazole-3-acetic acid, comprising the step of reacting 4-hydroxy-coumarin with hydroxylamine in the presence of sodium hydroxide, which avoids the direct use of metallic sodium; and is less hazardous. The process of the '325 application further provides a process for preparing a salt of benzisoxazole methane sulphonic acid, comprising the steps of 1) sulphonating 1,2-benzisoxazole-3-acetic acid using chlorosulphonic acid, in a solvent mixture that includes dichloroethane and sodium hydroxide; and 2) isolating the salt of benzisoxazole methane sulphonic acid. In the '325 application method, the sulphonation involves the use of chlorosulphonic acid in large quantities, which is used as reactant as well as a solvent. Therefore, a major impurity formed is the disulphonated product.

PCT application WO 03/072552 (“the '552 application”) discloses a process for manufacturing benisoxazole methane sulphonic acid chloride (BOS-Cl) and a process for preparing Zonisamide from BOS-Cl. The process disclosed in the '552 application comprises the steps shown below:

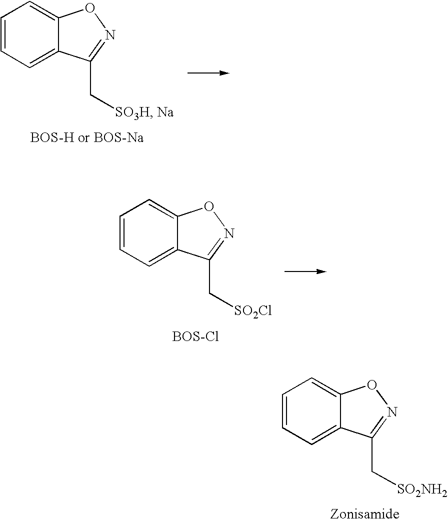

The process disclosed in the '552 application starts with a) chlorination of 1,2-benzisoxazole-3-methanesulphonic acid, salts or esters thereof; with thionyl chloride in an organic solvent and/or in the presence of a catalyst to form BOS-Cl; and b) amidation of BOS-Cl in the presence of ammonia selected from the group consisting of aqueous ammonia in a biphasic system, masked ammonia or dry ammonia to provide Zonisamide.

There us a need for an economical and industrially advantageous method for the manufacture of Zonisamide.

### SUMMARY OF THE INVENTION

The present invention to provides a process for the manufacture of an anti-epileptic agent, Zonisamide (Formula I). The process includes reaction of crystalline 1,2-benzisoxazole-methane-sulfonic acid sodium salt (BOS-Na), associated with NaCl, (also referred to herein as 1,2-benzisoxazole-methane-sulfonic acid sodium salt:sodium chloride or BOS-Na: NaCl) Formula II. Compounds with Formula I and Formula II have the following stuctures:

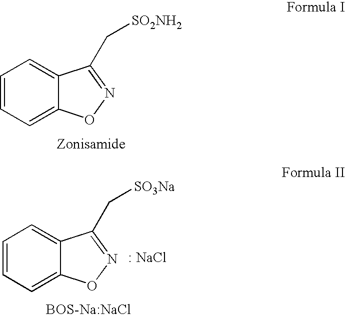

In a first embodiment, crystalline 1,2-Benzisoxazole-3-methane sodium sulphonate:NaCl (BOS-Na:NaCl), Formula II, synthesized from 4-hydroxycoumarin is isolated. The first step of the synthesis involves the reaction of 4-hydroxycoumarin with hydroxylamine hydrochloride in the presence of a methanolic solution of sodium methoxide to form 1,2-Benzisoxazole-3-acetic acid (BOA).

The second step of the synthesis comprises sulphonation of 1,2-Benisoxazole-3-acetic acid with chlorosulfonic acid dioxane complex. This reaction is carried out in ethylene chloride at 75 to 85° C. for 5 to 8 hrs. The reaction mixture is treated with 25% sodium hydroxide solution, and poured slowly into refluxing acetone. This provides crystalline BOS-Na:NaCl. HPLC purity=95%.

In another embodiment the crystalline BOS-Na:NaCl is heated with phosphorous oxychloride at 70-80° C. for 6 to 8 hours. After the reaction is complete excess of POCl₃ is removed under vacuum. The remaining mixture is taken up in ethyl acetate and treated with anhydrous ammonia gas at low temperature to provide crude 1,2-Bezisoxazole-3-methanesulphonamide.

The reaction, after re-crystallization of the product in methanol provides pure Zonisamide as white crystalline solid. HPLC purity=99%.

### DETAILED DESCRIPTION OF THE INVENTION

Zonisamide is an active anti-epileptic agent possessing anti-convulsant and anti-neurotoxic effects. The present invention provides a process for the manufacture of sodium chloride associated 1,2-Benzisoxazole-3-methane sodium sulphonate (BOS-Na:NaCl). The BOS-Na:NaCl thus prepared is directly converted to 1,2-Benzisoxazole-3-methanesulphonamide in good yield.

According to one embodiment the present invention, 1,2-Benzisoxazole-3-methanesulphonamide is prepared according the following synthetic reaction schemes:

(a) Reaction of 4-hydroxycoumarin and hydroxylamine hydrochloride

1,2-Benzisoxazole-3-acetic acid (BOA) is prepared by reacting 4-Hydroxy-coumarin with NH₂OH.HCl in the presence of sodium methoxide in alcoholic solution as shown in Scheme III, below:

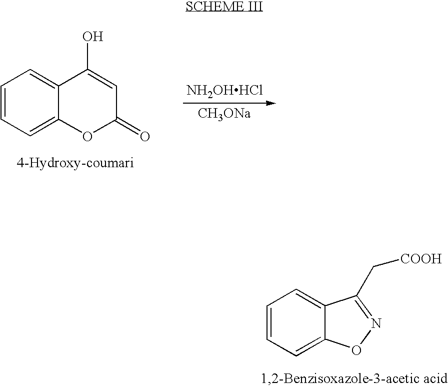

4-Hydroxymethylcoumarin is dissolved in methanol and hydroxylamine hydrochloride and sodium methoxide are added. The molar excess of hydroxylamine hydrochloride and sodium methoxide used in this reaction is typically from about 1 to about 4 fold, preferably between about 2 to 3 fold, relative to the 4-hydroxycoumarin.

The reaction mixture is heated at reflux until the reaction is complete. The reflux time for this reaction is between 5 to 10 hours. It is noted that as soon as the reaction is complete heating should be stopped to minimize the impurities. The reaction mixture is cooled and filtered to separate the inorganic solids from the solvent. The methanol layer is concentrated to provide a solid mass. The resultant solid is treated with aqueous sodium bicarbonate solution until alkaline and the alkaline mixture is extracted with an organic solvent, preferably diethyl ether. The organic layer, containing 2-hydroxyacetophenone oxime is discarded. Acidification of the aqueous layer with 2N hydrochloric acid will provide 1,2-benzisoxazole-3-acetic acid (BOA) in crystalline form, having a melting point of 122-124° C., and an HPLC purity between 95-98%.

(b) Sulfonation of 1,2-Benzisoxazole-3-acetic acid

1,2-Benzisoxazole-3-acetic acid is sulphonated under mild conditions using a chlorosulphonic acid:dioxane complex to provide 1,2-Benzisoxazole-3-methane sodium sulphonate:sodium chloride (BOS-Na:NaCl). The reaction process is outlined in Scheme IV, below:

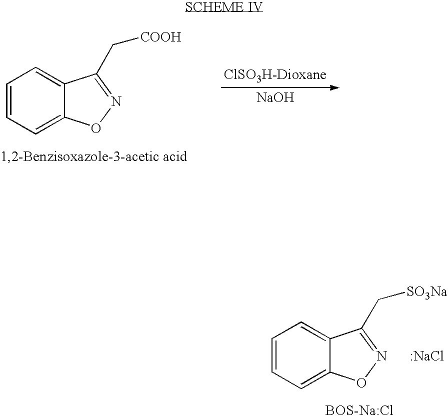

A mixture of 1,2-Benzisoxazole-3-acetic acid (BOA) and a chlorosulphonic acid: 1,4-dioxane complex is refluxed for 5-8 hours. (The chlorosulphonic acid: 1,4-dioxane complex mixture is prepared by admixing chlorosulphonic acid and 1,4-dioxane in a weight/weight ratio of about 0.5 to about 3.0 more preferably in a weight/weight ratio of about 1.0 to about 2.0 w/w ratio.) The use of a 1.3 to 2.0 mole ratio of chlorosulphonic acid, to 1,2-benzisoxazole-3-acetic acid, provides a reduced amount of disulphonated product. In the present invention the preferred solvent is an alkyl halide solvent, particularly ethylene dichloride.

U.S. Pat. No. 4,172,896 discloses the use of a complex of chlorosulphonic acid and 1,4-dioxane for sulphonation. However, the quantity of chlorosulphonic acid was significantly higher. This resulted in significant formation of disulphonated products. The removal of disulphonated products requires additional purification steps which costs time, solvent, and labor, etc.

In the present invention, due to controlled use of chlorosulphonic acid the formation of disulphonated products are not observed.

The sulphonated product, obtained in ethylene dichloride, is extracted with water. The organic layer is discarded and the aqueous layer is treated with 25% caustic lye solution. The aqueous reaction mixture is partially concentrated and it is poured into warm non-solvent such as, for example, acetone. The sodium salt of benzisoxazole methane sulphonic acid is obtained in solid form and is isolated by filtration and dried.

The product isolated, 1,2-benzisoxazole-3-methane sodium sulphonate, is associated with sodium chloride (BOS-Na:NaCl). This is confirmed by their characteristic X-Ray powder diffraction and IR absorption frequencies.

The present invention provides BOS-Na:Cl, characterized by X-Ray Powder Diffraction main peaks at about 18.64, 19.02, 19.80, 22.62, 23.14, 23.58, 25.52, 28.02, 29.00, 31.80, 32.10, 32.38, 32.96, 37.84, 38.28 and 38.60, ±0.2.

The IR Spectrum of BOS-Na:NaCl is characterized by peaks at about 3455, 1630, 1136, 955, 747, 638 and 619 cm⁻¹.

U.S. Patent Application No. 2003/0144527 A1 discloses different polymorphic forms of 1,2-Benzisoxazole-3 methane sodium sulphonate (BOS-Na). The different polymorphic forms of the BOS-Na are characterized by their characteristic XRD and IR peak values.

However, in the products prepared according to the method of the present invention the IR and XRD peak values of BOS-Na:NaCl are very different from polymorphic forms I, II, III, IV and V of BOS-Na. The XRD and IR values of the BOS-Na prepared according to the method of the invention are compared from the values reported in U.S. Patent Application No. 2003/0144527 A1. The comparative XRD and IR values of the different polymorphic forms of BOS-Na and BOS-Na:NaCl are set forth in Table 1.

TABLE 1
XRD and IR peak values of different polymorphic forms of BOS-Na and
BOS-Na:NaCl (XRD and IR peak values are reported in 2θ and
cm−1 respectively).

| BOS-Na | | BOS-Na | | BOS-Na | | BOS-Na | | BOS-Na | | | |
|---|---|---|---|---|---|---|---|---|---|---|---|
| Form I | | Form II | | Form III | | Form IV | | Form V | | BOS-Na:Cl | |
| XRD | IR | XRD | IR | XRD | IR | XRD | IR | XRD | IR | XRD | IR |
| 5.0 | 3546 | 5.3 | 3597 | 5.0 | 3604 | 4.5 | 3431 | 6.7 | 3601 | 18.64 | 3455 |
| 15.7 | 3485 | 16.6 | 3535 | 5.3 | 3495 | 5.9 | 1567 | 10.9 | 3520 | 19.02 | 1630 |
| 16.5 | 3440 | 21.3 | 3496 | 15.7 | 3067 | 8.8 | 1416 | 16.1 | 1587 | 19.80 | 1136 |
| 17.3 | 1612 | 26.7 | 3067 | 17.8 | 2998 | 11.3 | 924 | 21.0 | 1055 | 22.62 | 955 |
| 18.6 | 1513 | | 2998 | 21.4 | 2951 | 16.3 | 862 | 21.2 | 793 | 23.14 | 747 |
| 19.1 | 1439 | | 2951 | | 1605 | 16.9 | 586 | 22.2 | 753 | 23.58 | 638 |
| 19.7 | 1410 | | 1606 | | 1516 | 19.0 | | | | 25.52 | 619 |
| 21.5 | 1382 | | 1516 | | 1438 | 22.5 | | | | 28.02 | |
| 22.8 | 1234 | | 1438 | | 1382 | 23.9 | | | | 29.00 | |
| 23.2 | 1199 | | 1382 | | 1215 | 24.7 | | | | 31.80 | |
| 23.5 | 1048 | | 1213 | | 1136 | 25.0 | | | | 32.10 | |
| 24.3 | 918 | | 1064 | | 1065 | 26.8 | | | | 32.38 | |
| | 855 | | 1055 | | 1052 | 28.1 | | | | 32.96 | |
| | 760 | | 743 | | 777 | 29.7 | | | | 37.84 | |
| | 669 | | 663 | | 747 | 30.9 | | | | 38.28 | |
| | 593 | | 588 | | 696 | 32.6 | | | | 38.60 | |
| | | | 541 | | 588 | 33.6 | | | | | |
| | | | 522 | | 521 | 35.5 | | | | | |
| | | | | | | 36.6 | | | | | |

The BOS-Na isolated by acetone precipitation is associated with sodium chloride. Typically, a characteristic XRD peak of pure sodium chloride is observed at (2θ)31.82°. However a strong peak at (2θ) 31.80° is observed in BOS-Na:NaCl which provides evidence of the association of sodium chloride with BOS-Na.

(c) Chlorination and Amidation Reactions

BOS-Na:NaCl is converted to Zonisamide after the steps of converting the BOS-Na to an acid chloride, followed by amidation, as shown in Scheme IV below:

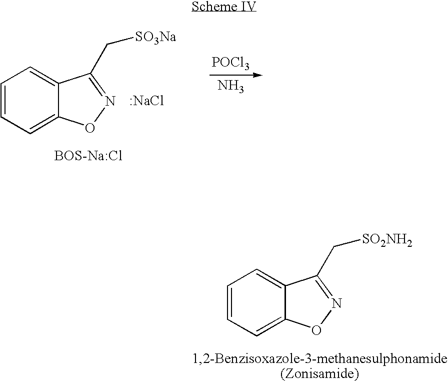

The product isolated from step (b), 1,2-Benzisoxazole-3-methane sodium sulphonate:sodium chloride (BOS-Na:NaCl) is converted to the corresponding acid chloride without further purification using phosphorous oxychloride (POCl₃) at a temperature of about 60 to 80° C., for about 4 to about 10 hours. The ratio of POCl₃ to BOS-Na:NaCl, used in this reaction is about a 3 times excess (by weight). The excess of phosphorous oxychloride present in the reaction mixture is removed by vacuum distillation to provide crude 1,2-benzisoxazole-3-methanesulfonyl chloride. Ethyl acetate (solvent) is added to the mixture and anhydrous ammonia is passed through the mixture to afford crude Zonisamide. The crude Zonisamide is re-crystallized in methanol to obtain white crystalline solid. After drying at 60-70° C. under vacuum dry crystalline Zonisamide having meting point 160-164° C. HPLC Purity=99% is obtained.

Elemental analysis of the isolated Zonisamide is shown below:

Zonisamide

| Elements | Calculated | Found |
|---|---|---|
| Nitrogen | 13.2 | 13.73 |
| Carbon | 45.28 | 45.70 |
| Hydrogen | 03.77 | 03.50 |
| Sulfur | 15.09 | 14.38 |

The ¹H NMR of Zonisamide has the following peaks (ppm relative to d6-DMSO, integration values in parentheses): 4.9 (2H), 7.25 (2H) and 7.4-8.0 (4H).

The IR spectrum of Zonisamide prepared is characterized by the peaks at about 3322.34, 3165.79, 3084.01, 2990.54, 2946.34, 1564.32, 1516.68, 1384.28, 1339.46, 1151.50, 1131.93, 936.72, 916.78, 869.08, 763.46, 747.39, 660.45 and 557.83 cm⁻¹.

The X-Ray powder diffraction of Zonisamide prepared is characterized by the following peaks (2θ) at about 11.420, 13.400, 14.400, 14.720, 16.300, 18.520, 18.820, 19.720, 22.180, 22.520, 22.920, 23.900, 24.260, 25.620, 26.560, 27.220, 27.660, 28.780, 29.160, 29.660, 29.960, 31.120, 31.700, 32.040, 32.920, 33.400, 34.180, 34.680, 35.200, 36.460, 37.180, 37.500, 38.220 and 38.860°.

The molecular weight of the Zonaside (MW 212) prepared is characterized by scanning mass of the finally crystallized product, which is found to be exactly the same as the theoretical value.

The following examples are offered to aid in understanding of the present invention and are not to be construed as limiting the scope thereof.

### EXAMPLE 1

### 1,2-Benisoxazole-3-acetica acid

A methanolic solution of 4-hydroxycoumarin (59 Kg, 314.19 moles) is heated at reflux with hydroxylamine hydrochloride (75.98 Kg, 1093.23 moles) and sodium methoxide 26.18% w/v solution (225.38 Ltr, 1092.27 mole) for 5 to 10 hours. After the reaction is complete, the inorganic solid is filtered off, and the filtrate, containing 1,2-Benzisoxazole-3-acetic acid in methanol, is concentrated to afford a solid residue. The residue is made alkaline with sodium bicarbonate and extracted with diethyl ether. 2-Hydroxyacetophenone oxime is found, in the ether extract and is discarded. Upon further, acidification of the aqueous layer with 2N hydrochloric acid, 1,2-Benzisoxazole-3-acetic acid (49.18 Kg) is obtained as a crystalline solid with melting point 122 to 124° C. and having HPLC purity about 95 to 98%.

### EXAMPLE 2

### 1,2-Benzisoxazole-3-methane sodium sulphonate (BOS-Na:NaCl)

A mixture of 1,2-Benzisoxazole-3-acetic acid (49.18 Kg, 277.85 moles) and chlorosulphonic acid: 1,4-dioxane complex (132.50 L) [prepared by addition at low temperature 1,4-dioxane (105.30 Kg) in chlorosulphonic acid (53.50 Kg, 459.15 mole)] in dichloroethane is refluxed for 5 to 8 hours.

After the reaction is completed, the mixture is cooled to room temperature and quenched by addition of chilled water. The organic layer separated from the aqueous layer and discarded. The aqueous layer is made alkaline with 25% sodium hydroxide solution and is partially concentrated, to 150 to 200 L. The aqueous solution is added to acetone at reflux. The solid mass formed is filtered and dried at 65 to 70° C. under vacuum for 6 to 8 hours to provide 90 Kg of product having a melting point of 266 to 270° C. and HPLC purity of not less than 95%. This product is associated with sodium chloride, which is used in the next stage to manufacture Zonisamide without further purification.

### EXAMPLE 3

### 1,2-Benzisoxazole-3-methanesulphonamide (Zonisamide)

1,2-Benisoxazole-3-methane sodium sulphonate (90 Kg) associated with sodium chloride and phosphorous oxychloride (282 Kg) is heated at 70 to 80° C. temperature for 6 to 8 hours to form an acid chloride. The excess POCl₃ is distilled under vacuum and the remaining material is taken up in ethyl acetate. The product in ethyl acetate is treated with anhydrous ammonia gas at low temperature to provide crude Zonisamide. The crude product is re-crystallized in methanol to provide pure Zonisamide (30 Kg) as a white crystalline solid. The isolated product, dried at 60 to 70° C. under vacuum for 8 to 10 hours, has a melting point of 160 to 164° C. and HPLC purity=99%.

All patents, patent applications, and literature cited in the specification are hereby incorporated by reference in their entirety. In the case of any inconsistencies, the present disclosure, including any definitions therein will prevail.

We claim:

1. A process for the manufacturing a compound of Formula I 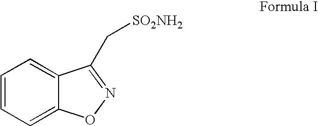
comprising:
(a) subjecting a compound of the Formula II 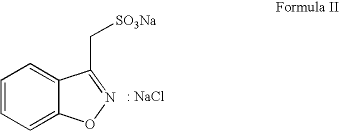
to a chlorination reaction with a chlorinating agent, followed by an amidation reaction; and
(b) isolating the 1,2-benzisoxazole-3-methanesulphonamide compound of formula I.

2. The process of claim 1, wherein chlorinating agent is phosphorous oxychloride.

3. The process of claim 1, wherein the temperature for the chlorination reaction is from about 60 to about 90° C.

4. The process of claim 1, wherein the reaction time for the chlorination reaction is from about 4 to about 12 hours.

5. The process of claim 1, wherein the excess of phosphorous oxychloride is removed by vacuum distillation.

6. The process of claim 1, wherein the amidation reaction is carried out in an organic solvent.

7. The process of claim 6, wherein the organic solvent is an aliphatic ester.

8. The process of claim 7, wherein the aliphatic ester is ethyl acetate.

9. The process of claim 1, wherein anhydrous ammonia gas is used for the amidation reaction.

10. The process of claim 1, further comprising re-crystallizing the 1,2-benzisoxazole-3-methanesulphonamide from step (b) in a protic solvent.

11. The process of claim 10, wherein the protic solvent is methanol.

12. The process of claim 11, wherein the re-crystallized 1,2-benzisoxazole-3-methanesulphonamide is greater than about 99% purity.

13. A process for the preparation of 1,2-benzisoxazole-3-methane sodium sulphonate:sodium chloride having Formula II: 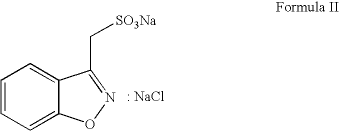
comprising,
(a) reacting 4-hydroxycoumarin with hydroxylamine hydrochloride in the presence of a base to provide 1,2-benzisoxazole-3-acetic acid; and
(b) reacting the 1,2-benzisoxazole-3-acetic acid with a chlorosulfonic acid complex to provide 1,2-benzisoxazole-3-methane sodium sulphonate:NaCl.

14. The process of claim 13, further comprising the step of converting the 1,2-benzisoxazole-3-methane sodium sulphonate:NaCl to 1,2-benisoxazole-3-sulfonamide.

15. The process of claim 13, wherein the base is sodium methoxide.

16. The process of claim 13, wherein the reaction of step (a) is carried out in methanol.

17. The process of claim 13, wherein the chlorosulfonic acid complex is a complex of chlorosulfonic acid and 1,4-dioxane.

18. The process of claim 17, wherein the molar ratio of 1,2-benzisoxazole-3-acetic acid and chlorosulfonic acid complex is from about 1 to about 3.

19. The process of claim 18, wherein the molar ratio of 1,2-benzisoxazole-3-acetic acid and chlorosulfonic acid complex is from about 1 to about 2.

20. The process of claim 13, wherein the reaction of step (a) is carried out in a halo-aliphatic solvent.

21. The process of claim 20, wherein the halo-aliphatic solvent is dichloroethane.

22. The process of claim 17, wherein the reaction time is from about 2 to about 10 hours.

23. The process of claim 22, wherein reaction temperature is the reflux temperature of the solvent.

24. The process of claim 13, further comprising isolating the 1,2-benzisoxazole methane sulphonic acid sodium salt (BOS-Na) from a mixture of water and acetone.

25. The process of claim 24, wherein 1,2-benzisoxazole methane sulphonic acid sodium salt is associated with sodium chloride (BOS-Na:NaCl).

26. The process of claim 25, wherein the BOS-Na:NaCl is isolated in crystalline form having an HPLC purity greater than 95%.

27. The process of claim 26, wherein crystalline BOS-Na:NaCl is characterized by an X-Ray powder diffraction (XRD) having the peaks at about 11.420, 13.400, 14.400, 14.720, 16.300, 18.520, 18.820, 19.720, 22.180, 22.520, 22.920, 23.900, 24.260, 25.620, 26.560, 27.220, 27.660, 28.780, 29.160, 29.660, 29.960, 31.120, 31.700, 32.040, 32.920, 33.400, 34.180, 34.680, 35.200, 36.460, 37.180, 37.500, 38.220 and 38.860±0.2 degree two theta.

28. The process of claim 26, wherein the product has an Infra Red Spectroscopy (IR) spectrum having peaks at about 3322.34, 3165.79, 3084.01, 2990.54, 2946.34, 1564.32, 1516.68, 1384.28, 1339.46, 1151.50, 1131.93, 936.72, 916.78, 869.08, 763.46, 747.39, 660.45 and 557.83 cm⁻¹.

29. The process of claim 14, wherein the BOS-Na:NaCl is used directly after isolating from step (b) of claim 13.
